SEQUENCE LISTING (4 sequences)

SEQ ID NO: 1 - DNA, 83 bp, Artificial
  tracr mate sequence and tracr sequence
gttttagagctagaaatagcaagttaaaataaggctagtccgttatcaacttgaaaaagtggcaccgagtcggtgcttttttt

SEQ ID NO: 2 - protein, 1368 aa, Streptococcus pyogenes [sequence omitted]

SEQ ID NO: 3 - DNA, 23 bp, Artificial
  reporter
ggggccactagggacaggattgg

SEQ ID NO: 4 - DNA, 23 bp, Artificial
  reporter
aagagagacagtacatgccctgg

(19) United States
(12) Patent Application Publication
(10) Pub. No.: US 2018/0230450 A1
(43) Pub. Date: Aug. 16, 2018

(54) Cas9 Genome Editing and Transcriptional Regulation

(71) Applicant: President and Fellows of Harvard College, Cambridge, MA (US)

(72) Inventors: Alejandro Chavez, Sharon, MA (US); Marcelle Tuttle, Somerville, MA (US)

(21) Appl. No.: 15/749,168

(22) PCT Filed: Aug. 3, 2016
(86) PCT No.: PCT/US16/45254

§ 371 Date: Jan. 31, 2018

Related U.S. Application Data
(60) Provisional application No. 62/200,303, filed on Aug. 3, 2015.

(51) Int. Cl. C12N 15/10; C12N 15/63; C12N 15/90; C12N 9/22

(52) U.S. Cl. CPC C12N 15/102; C12N 15/63; C12N 2310/20; C12N 9/22; C12N 15/90

(57) ABSTRACT

CRISPR/Cas Systems are provided where two or more guide RNAs having different spacer sequence lengths direct an enzymatically active Cas9 having a transcriptional regulator attached thereto to either cut a target nucleic acid or regulate expression of a target nucleic acid.

### RELATED APPLICATION DATA

This application claims priority to U.S. Provisional Application No. 62/200,303 filed on Aug. 3, 2015 which is hereby incorporated herein by reference in its entirety for all purposes.

### STATEMENT OF GOVERNMENT INTERESTS

This invention was made with government support under P50 HG005550 awarded by US National Institutes of Health National Human Genome Research Institute, DE-FG02-02ER63445 awarded by the US Department of Energy and 5T32CA009216-34 awarded by the National Cancer Institute. The government has certain rights in the invention.

### BACKGROUND

The CRISPR type II system is a recent development that has been efficiently utilized in a broad spectrum of species. See Friedland, A. E., et al., Heritable genome editing in C. elegans via a CRISPR-Cas9 system. Nat Methods, 2013. 10(8): p. 741-3, Mali, P., et al., RNA-guided human genome engineering via Cas9. Science, 2013. 339(6121): p. 823-6, Hwang, W. Y., et al., Efficient genome editing in zebrafish using a CRISPR-Cas system. Nat Biotechnol, 2013, Jiang, W., et al., RNA-guided editing of bacterial genomes using CRISPR-Cas systems. Nat Biotechnol, 2013, Jinek, M., et al., RNA-programmed genome editing in human cells. elife, 2013. 2: p. e00471, Cong, L., et al., Multiplex genome engineering using CRISPR/Cas systems. Science, 2013. 339(6121): p. 819-23, Yin, H., et al., Genome editing with Cas9 in adult mice corrects a disease mutation and phenotype. Nat Biotechnol, 2014. 32(6): p. 551-3. CRISPR is particularly customizable because the active form consists of an invariant Cas9 protein and an easily programmable guide RNA (gRNA). See Jinek, M., et al., A programmable dual-RNA-guided DNA endonuclease in adaptive bacterial immunity. Science, 2012. 337(6096): p. 816-21. Of the various CRISPR orthologs, the Streptococcus pyogenes (Sp) CRISPR is the most well-characterized and widely used. The Cas9-gRNA complex first probes DNA for the protospacer-adjacent motif (PAM) sequence (−NGG for Sp Cas9), after which Watson-Crick base-pairing between the gRNA and target DNA proceeds in a ratchet mechanism to form an R-loop. Following formation of a ternary complex of Cas9, gRNA, and target DNA, the Cas9 protein generates two nicks in the target DNA, creating a blunt double-strand break (DSB) that is predominantly repaired by the non-homologous end joining (NHEJ) pathway or, to a lesser extent, template-directed homologous recombination (HR). CRISPR methods are disclosed in U.S. Pat. No. 9,023,649 and U.S. Pat. No. 8,697,359. See also, Fu et al., Nature Biotechnology, Vol. 32, Number 3, pp. 279-284 (2014). Additional references describing CRISPR-Cas9 systems including nuclease null variants (dBas9) and nuclease null variants functionalized with effector domains such as transcriptional activation domains or repression domains include J. D. Sander and J. K. Joung, Nature biotechnology 32 (4), 347 (2014); P. D. Hsu, E. S. Lander, and F. Zhang, Cell 157 (6), 1262 (2014); L. S. Qi, M. H. Larson, L. A. Gilbert et al., Cell 152 (5), 1173 (2013); P. Mali, J. Aach, P. B. Stranges et al., Nature biotechnology 31 (9), 833 (2013); M. L. Maeder, S. J. Linder, V. M. Cascio et al., Nature methods 10 (10), 977 (2013); P. Perez-Pinera, D. D. Kocak, C. M. Vockley et al., Nature methods 10 (10), 973 (2013); L. A. Gilbert, M. H. Larson, L. Morsut et al., Cell 154 (2), 442 (2013); P. Mali, K. M. Esvelt, and G. M. Church, Nature methods 10 (10), 957 (2013); and K. M. Esvelt, P. Mali, J. L. Braff et al., Nature methods 10 (11), 1116 (2013).

### SUMMARY

Embodiments of the present disclosure are directed to methods of using an enzymatically active Cas9, such as a Cas9 nuclease or nickase, optionally having a functional group attached thereto, and a guide RNA which is used to guide the enzymatically active Cas9 with the functional group attached thereto to a target nucleic acid. According to one aspect, when a functional group is attached to the enzymatically active Cas9, the functional group is directed to a target nucleic acid to perform the desired function on the target nucleic acid, such as transcriptional regulation. Also, it is to be understood that transcriptional regulation can also be accomplished according to methods described herein where an enzymatically active Cas9 is used without any attached functional group and transcriptional regulation is accomplished by inhibition of transcription due to the Cas9 forming a complex at the target nucleic acid and without cutting the target nucleic acid. According to one aspect, the guide RNA includes a spacer sequence having a length sufficient to bind to a target nucleic acid and form a complex with the enzymatically active Cas9 optionally with the functional group attached thereto, but insufficient for the enzymatically active Cas9 to function to cut or nick the target nucleic acid. Without wishing to be bound by scientific theory, based on the length of the spacer sequence of the guide RNA, the enzymatic activity of the enzymatically active Cas9 is blocked or prevented or otherwise inhibited, and the otherwise enzymatically active Cas9 is effectively rendered a nuclease null Cas9. According to this aspect, the functional group when attached to the enzymatically active Cas9 performs the desired function of the functional group, as the enzymatically active Cas9 nuclease does not function to cut or nick the target nucleic acid. Without wishing to be bound by any scientific theory, the enzymatically active Cas9 optionally with the functional group attached thereto forms a co-localization complex with the guide RNA and the target nucleic acid, however, the length of the spacer sequence of the guide RNA results in an inability of the Cas9 to cleave the target nucleic acid substrate.

Embodiments of the present disclosure are directed to methods of using a enzymatically active Cas9, such as a Cas9 nuclease or nickase, optionally having a functional group attached thereto and a guide RNA with a spacer sequence having a length sufficient to bind to a target nucleic acid and to form a complex with the enzymatically active Cas9 optionally with the functional group attached thereto, and sufficient to allow the enzymatically active Cas9 to function as a nuclease or nickase with respect to the target nucleic acid. According to one aspect, the functional group when optionally attached to the enzymatically active Cas9 does not perform the desired function of the functional group, as the target nucleic acid is either cut or nicked by the enzymatically active Cas9.

Embodiments of the present disclosure are directed to methods of using a enzymatically active Cas9, such as a Cas9 nuclease or nickase, optionally having a functional group attached thereto, a first guide RNA with a spacer sequence having a length sufficient to bind to a first target nucleic acid and form a complex with the enzymatically active Cas9 optionally having the functional group attached thereto, but insufficient to allow the enzymatically active Cas9 to function as a nuclease or nickase with respect to the first target nucleic acid, and a second guide RNA with a spacer sequence having a length sufficient to bind to a second target nucleic acid and form a complex with the enzymatically active Cas9 optionally having the functional group attached thereto, such as a Cas9 nuclease or nickase, and sufficient to allow the enzymatically active Cas9 to function as a nuclease or nickase with respect to the second target nucleic acid. According to this aspect, the enzymatically active Cas9 when complexed with the first guide RNA at the first target nucleic acid will function as a nuclease null Cas9 to deliver a functional group if present to the first target nucleic acid and the enzymatically active Cas9 when complexed with the second guide RNA at the second target nucleic acid will also function as a nuclease or nickase to either cut or nick the second target nucleic acid.

Aspects of the present disclosure are directed to programmable genome editing as an enzymatically active Cas9 can be used to cut or nick a target nucleic acid by selection of a first guide RNA sequence and the same Cas9 can be effectively rendered nuclease null by selection of a second guide RNA sequence which allows the Cas9 to complex at the target nucleic acid sequence but not cut or nick the target nucleic acid sequence. Such complex formation can have an inhibitory effect on transcription and therefore can regulate gene expression without using a separate transcription regulator functional group.

Aspects of the present disclosure are directed to programmable genome editing and use of a functional group, such as a transcriptional regulator, using the same species of enzymatically active Cas9 having the functional group attached thereto. Methods described herein are directed to the use of a single species of enzymatically active Cas9 having a transcriptional regulator attached thereto which can be simultaneously used for genome editing of target nucleic acids and transcriptional regulation of genes, based on the spacer sequence length of the particular guide RNA. The length of the guide RNA spacer sequence determines the ability of the enzymatically active Cas9 species having a functional group (such as a transcriptional regulator) attached thereto to either (1) function to deliver the functional group to a target nucleic acid so that the functional group can perform its desired function or (2) function as an enzyme to cut or nick a target nucleic acid.

According to certain aspects, the enzymatically active Cas9 optionally having a functional group attached thereto is present within a cell and two or more guide RNAs are provided to a cell in series or simultaneously wherein each guide RNA is designed to complex with the enzymatically active Cas9 optionally having a functional group attached thereto at respective target nucleic acid sites or sequences. Each guide RNA has a spacer sequence length that determines whether the enzymatically active Cas9 optionally having a functional group attached thereto will function as either an enzyme to cut or nick a nucleic acid or as a nuclease null Cas9 to form a complex at the target nucleic acid and deliver the functional group if present to a target nucleic acid so that the functional group may perform its function on a target nucleic acid. In this manner, enzymatically active Cas9 optionally having a functional group attached thereto may first be used to cut or nick a nucleic acid and then be used to deliver a functional group if present to a nucleic acid sequence so that the functional group may perform the function or vice versa. According to one aspect, a plurality of guide RNAs may be used to target the enzymatically active Cas9 optionally having a functional group attached thereto, such as a single species of an enzymatically active Cas9 optionally having a functional group attached thereto, to a plurality of different target nucleic acid sites to perform either cutting or nicking or functional group delivery.

When the enzymatically active Cas9 optionally having a functional group attached thereto is used for cutting or nicking a target nucleic acid, methods described herein contemplate the use of one or more donor nucleic acids that may be inserted into one or more cut or nick sites through homologous recombination or nonhomologous end joining. Accordingly, methods described herein are directed to methods of genome editing using the enzymatically active Cas9 optionally having a functional group attached thereto and also methods of targeting a functional group when present to a target nucleic acid to perform the function of the functional group using the enzymatically active Cas9 having a functional group attached thereto. One of skill will readily understand that the utility of the enzymatically active Cas9 optionally having a functional group attached thereto is determined by the spacer sequence length of the guide RNA and whether the guide RNA has a spacer sequence length that facilitates enzymatic activity of the enzymatically active Cas9 or not.

According to certain aspects, a guide RNA that allows enzymatic activity of the enzymatically active Cas9 having a functional group attached thereto includes a spacer sequence having an exemplary nucleotide length of between 25 and 15 nucleotides, such as between 20 and 16 nucleotides. According to certain aspects, a guide RNA that inhibits enzymatic activity of the enzymatically active Cas9 optionally having a functional group attached thereto includes a spacer sequence having an exemplary nucleotide length of between 8 and 14 nucleotides.

According to certain aspects, a guide RNA includes a spacer sequence and a tracr mate sequence forming a crRNA, as is known in the art. According to certain aspects, a tracr sequence, as is known in the art, is also used in the practice of methods described herein. According to one aspect, the tracr sequence and the crRNA sequence may be separate or connected by the linker, as is known in the art.

According to one aspect, the enzymatically active Cas9 with the functional group attached thereto is provided to the cell by introducing into the cell a first foreign nucleic acid encoding the enzymatically active Cas9 protein and wherein the guide RNA is provided to the cell by introducing into the cell a second foreign nucleic acid encoding the guide RNA, wherein the guide RNA and the enzymatically active Cas9 with the functional group attached thereto are expressed, and wherein the guide RNA and the enzymatically active Cas9 with the functional group attached thereto co-localize to the target nucleic acid. According to one aspect, the enzymatically active Cas9 protein is a fully enzymatic Cas9 protein as is known in the art or a Cas9 protein nickase as is known in the art. According to one aspect, the cell is in vitro, in vivo or ex vivo. According to one aspect, the cell is a eukaryotic cell or prokaryotic cell. According to one aspect, the cell is a bacteria cell, a yeast cell, a fungal cell, a mammalian cell, a plant cell or an animal cell. According to one aspect, the target nucleic acid is genomic DNA, mitochondrial DNA, plastid DNA, viral DNA, exogenous DNA or cellular RNA.

According to one aspect, a cell is provided which includes an enzymatically active Cas9 with a functional group attached thereto and at least one guide RNA including a spacer sequence and a tracr mate sequence forming a crRNA and a tracr sequence and wherein the guide RNA and the enzymatically active Cas9 with a functional group attached thereto are members of a co-localization complex for the target nucleic acid. According to an additional aspect, at least two guide RNAs are provided with a first guide RNA including an exemplary spacer sequence between 25 and 15 nucleotides in length and a second guide RNA having an exemplary spacer sequence between 14 and 8 nucleotides in length. According to one aspect, the cell is a eukaryotic cell or prokaryotic cell. According to one aspect, the cell is a bacteria cell, a yeast cell, a fungal cell, a mammalian cell, a plant cell or an animal cell.

According to one aspect, a genetically modified cell is provided including a first foreign nucleic acid encoding an enzymatically active Cas9 optionally having a functional group attached thereto and a second foreign nucleic acid encoding a guide RNA including a spacer sequence and a tracr mate sequence forming a crRNA and a tracr sequence and wherein the guide RNA and the enzymatically active Cas9 optionally having a functional group attached thereto are members of a co-localization complex for the target nucleic acid. According to an additional aspect, one or more foreign nucleic acids are provided encoding at least two guide RNAs with a first guide RNA having an exemplary spacer sequence between 25 and 15 nucleotides in length and a second guide RNA having an exemplary spacer sequence between 14 and 8 nucleotides in length. According to one aspect, the cell is a eukaryotic cell or prokaryotic cell. According to one aspect, the cell is a bacteria cell, a yeast cell, a fungal cell, a mammalian cell, a human cell, a plant cell or an animal cell.

According to one aspect, a functional group may be any desired functional group as known to those of skill in the art. An exemplary functional group may be an effector domain, such as a transcriptional activator or transcriptional repressor, or a detectable group, such as fluorescent protein, or a binding functional group, such as an apatamer, which can be used to bind to a desired functional group or a nuclear localization signal, which can be used to deliver the Cas9 to a nucleus.

Further features and advantages of certain embodiments of the present invention will become more fully apparent in the following description of embodiments and drawings thereof, and from the claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The foregoing and other features and advantages of the present embodiments will be more fully understood from the following detailed description of illustrative embodiments taken in conjunction with the accompanying drawings in which:

FIGS. 1A-1D are directed to activation and cutting of endogenous genes in 293T cells. FIGS. 1A-1C are graphs depicting data of RNA expression and mutagenesis analysis of the genes ACTC1, MIAT, and TTN, respectively. Each sample was transfected with the indicated Cas9 construct and gRNA of particular length. Data indicate the mean∓s.e.m (n=2 independent transfections). *P<0.05 when compared to guide control for activation experiments. FIG. 1D are graphs depicting data of multiplexed activation and cutting of the genes ACTC1, MIAT, and TTN. The indicated constructs were transfected with a 20 nt ACTC1 gRNA and 14 nt MIAT and TTN gRNAs simultaneously. Data indicate the mean∓s.e.m (n=2 independent transfections). *P<0.05 when compared to guide control for activation experiments.

FIGS. 2A-2B depict data directed to activation and cutting of a transcriptional reporter using gRNAs with progressively shorter 5′ end length. FIG. 2A is a graph of data showing deletion analysis of truncated gRNAs on a synthetic transcriptional reporter. Samples were transfected with the indicated Cas9 construct and gRNA length. Data indicates the mean s.e.m (n=2 independent transfections). FIG. 2B is a graph depicting quantification of activation for truncated gRNAs via a fluorescent transcriptional reporter. Data indicates the mean s.e.m (n=2 independent transfections).

FIG. 3A-3B is directed to activation and cutting of a transcriptional reporter using orthogonal Cas9 proteins. FIG. 3A shows graphs of data of deletion analysis of truncated gRNAs on a synthetic transcriptional reporter using SA and ST1-Cas9. Samples were transfected with the indicated Cas9 construct and gRNA length. Data indicates the mean∓s.e.m (n=2 independent transfections). FIG. 3B shows graph of data of quantification of activation for truncated gRNAs via a fluorescent transcriptional reporter using SA and ST1-Cas9. Data indicates the mean∓s.e.m (n=2 independent transfections)

FIG. 4 is directed to activation and cutting of endogenous HBG1 gene. Each sample was transfected with the indicated Cas9 construct and gRNA of particular length. Data indicate the mean∓s.e.m (n=2 independent transfections). *P<0.05 when compared to guide control for activation experiments.

FIG. 5 is directed to comparison of activation of Cas9-VPR targeted to three tdTomato reporters using 20 nt or 14 nt with single mismatches at each position. Single mismatch mutations are Watson-Crick transversions. −1 position is adjacent to the PAM sequence. tdTomato reporter activation by Cas9-VPR was measured using flow cytometry and values shown represent the ratio of activation signal observed from each gRNA relative to a fully matched gRNA. In other words, the 20 nt mismatched guides were normalized to the fully matched 20 nt gRNA and the 14 nt mismatched guides were normalized to the fully matched 14 nt gRNA. NM represents “no mismatches” or the fully matched gRNA samples. n=2 independent biological replicates. Data represent normalized median with error bars representing s.e.m.

FIG. 6A and FIG. 6B are directed to off-target expression analysis. FIG. 6A is directed to gene expression levels (log2TPM, Transcripts Per Million) in cells transfected with dCas9-VPR targeting indicated genes with guide RNAs of indicated lengths (y axis) vs. expression in cells transfected with guide RNA only (x axis). R indicates Pearson's correlation coefficient calculated for log-transformed values on all genes except the target. A pseudocount of 1 TPM was added to each gene before log transforming. Average of two biological replicates are shown. FIG. 6B are histograms showing the distribution of fold-changes in gene expression (activator/guide control). Genes were filtered to include only those with TPM>1. Average of two biological replicates shown.

FIG. 7 is a pictorial representation of FIG. 1D showing the expected behavior of Cas9-VPR. Cells were transfected with a 20 nt guide directed towards ACTC1, 14 nt guides directed towards MIAT and TTN and either Cas9-VPR or Cas9. The ACTC1 locus should be cut, while transcription occurs for the genes MIAT and ACTC1.

FIG. 8 are graphs depicting data of gRNA-mediated recruitment of an activator using Cas9 protein. The indicated constructs were transfected with a 20 nt ACTC1 gRNA and 14 nt MIAT and TTN gRNAs simultaneously along with the indicated Cas9 and/or AD. Data indicates the mean s.e.m (n=2 independent transfections). *P<0.05 when compared to guide control.

### DETAILED DESCRIPTION

Embodiments of the present disclosure are directed to the use of a CRISPR/Cas system and, in particular, an enzymatically active Cas9 protein optionally having a functional group attached thereto, and one or more guide RNAs which includes a spacer sequence, a tracr mate sequence and a tracr sequence. According to certain aspects, a guide RNA which facilities enzymatic activity of the Cas9 protein has an exemplary spacer sequence including between 25 and 15 nucleotides in length. According to certain aspects, a guide RNA which inhibits enzymatic activity of the Cas9 protein has an exemplary spacer sequence including between 14 and 8 nucleotides in length. According to certain methods, two or more or a plurality of guide RNAs may be used in the practice of certain embodiments based on whether one of skill desires the species of enzymatically active Cas9 protein optionally having a functional group attached thereto to cut or nick a desired nucleic acid or to deliver the functional group to a desired nucleic acid so that the functional group can perform the function.

The term spacer sequence is understood by those of skill in the art and may include any polynucleotide having sufficient complementarity with a target nucleic acid sequence to hybridize with the target nucleic acid sequence and direct sequence-specific binding of a CRISPR complex to the target sequence. A CRISPR complex may include the guide RNA and the Cas9 protein. The guide RNA may be formed from a spacer sequence covalently connected to a tracr mate sequence (which may be referred to as a crRNA) and a separate tracr sequence, wherein the tracr mate sequence is hybridized to a portion of the tracr sequence. According to certain aspects, the tracr mate sequence and the tracr sequence are connected or linked such as by covalent bonds by a linker sequence, which construct may be referred to as a fusion of the tracr mate sequence and the tracr sequence. The linker sequence referred to herein is a sequence of nucleotides, referred to herein as a nucleic acid sequence, which connect the tracr mate sequence and the tracr sequence. Accordingly, a guide RNA may be a two component species (i.e., separate crRNA and tracr RNA which hybridize together) or a unimolecular species (i.e., a crRNA-tracr RNA fusion, often termed an sgRNA).

Tracr mate sequences and tracr sequences are known to those of skill in the art, such as those described in US 2014/0356958. The tracr mate sequence and tracr sequence used in the present disclosure is N20 to N8-gttttagagctagaaatagcaagttaaaataaggctagtccgttatcaacttgaaaaagtggcaccgagtcggtgcttttttt with N20-8 being the number of nucleotides complementary to a target locus of interest.

According to certain aspects, the guide RNA spacer sequence length determines whether the enzymatically active Cas9 optionally having a functional group attached thereto will function to cut or nick the target nucleic acid or to act as a nuclease null Cas9 and deliver the functional group if present to the target nucleic acid so that the functional group can perform the desired function. A guide RNA having a spacer sequence length where the enzymatically active Cas9 will cut or nick the target nucleic acid may be termed an “enzymatic guide RNA” to the extent that such a guide RNA facilitates enzymatic activity of the Cas9. An enzymatic guide RNA has an exemplary spacer sequence length of 25 to 15 nucleotides. A guide RNA having a spacer sequence length where the enzymatically active Cas9 will function as a nuclease null Cas9 and may be termed a “nonenzymatic guide RNA” to the extent that such a guide RNA will inhibit enzymatic activity of the Cas9. A nonenzymatic guide RNA has an exemplary spacer sequence length of 14 to 8 nucleotides. It is to be understood that the enzymatically active Cas9 may still be referred to as such even though it is used with a nonenzymatic guide RNA and where the enzymatically active Cas9 does not cut or nick the target nucleic acid. The enzymatically active Cas9 can be programmed to cut or operate as a nuclease null Cas9 based on the selected spacer sequence length. It is to be understood that for particular target nucleic acids, an exemplary enzymatic guide RNA length or an exemplary nonenzymatic guide RNA length may include 1 or two nucleotides outside of the exemplary ranges described herein.

According to certain aspects, the tracr mate sequence is between about 17 and about 27 nucleotides in length. According to certain aspects, the tracr sequence is between about 65 and about 75 nucleotides in length. According to certain aspects, the linker nucleic acid sequence is between about 4 and about 6.

The functional group may be joined, fused, connected, linked or otherwise tethered, such as by covalent bonds, to the enzymatically active Cas9 protein using methods known to those of skill in the art.

Functional groups within the scope of the present disclosure include transcriptional modulators or effector domains known to those of skill in the art. Suitable transcriptional modulators include transcriptional activators. According to one aspect, the transcriptional regulator protein or domain upregulates expression of the target nucleic acid. Suitable transcriptional modulators include transcriptional repressors. According to one aspect, the transcriptional regulator protein or domain downregulates expression of the target nucleic acid. Exemplary transcriptional activators include VP64, VP16, VP160, VP48, VP96, p65, Rta, VPR, hsf1, and p300. Suitable transcriptional repressors include KRAB. Transcriptional activators and transcriptional repressors can be readily identified by one of skill in the art based on the present disclosure.

Functional groups within the scope of the present disclosure include detectable groups or markers or labels. Such detectable groups or markers or labels can be detected or imaged using methods known to those of skill in the art to identify the location of the target nucleic acid sequence. Indirect attachment of a detectable label or maker is contemplated by aspects of the present disclosure. Detectable labels or markers can be readily identified by one of skill in the art based on the present disclosure. Detectable groups include fluorescent proteins such as GFP, RFP, BFP, EYFP, sfGFP, mcherry, iRFP, citrine, morange, cerulean, mturquoise, EBFP, EBFP2, Azurite, mKalamal, ECFP, CYPET, mTurquoise2, YFP, Venus, and Ypet and the like. Other useful detectable groups include spytag, spycatcher, snap tags, biotin, streptavidin, and suntag and the like.

Functional groups within the scope of the present disclosure include binding functional groups which may function to bind to desired molecules. Such binding functional groups include aptamers ms2 to MCP, pp7 to PCP, com to Com binding protein, inteins, FKBP to FRB, pMAG to nMAG and Cry2 and the like.

According to one aspect, embodiments described herein include guide RNA having a length including the sum of the lengths of a spacer sequence, tracr mate sequence, tracr sequence, and linker sequence (if present). Accordingly, such a guide RNA may be described by its total length which is a sum of its spacer sequence, tracr mate sequence, tracr sequence, and linker sequence. According to this aspect, all of the ranges for the spacer sequence, tracr mate sequence, tracr sequence, and linker sequence (if present) are incorporated herein by reference and need not be repeated. One of skill will readily be able to sum each of the portions of a guide RNA to obtain the total length of the guide RNA sequence. Aspects of the present disclosure are directed to methods of making such guide RNAs as described herein by expressing constructs encoding such guide RNA using promoters and terminators and optionally other genetic elements as described herein.

According to certain aspects, the guide RNA and the enzymatically active Cas9 optionally having a functional group attached thereto which interacts with the guide RNA are foreign to the cell into which they are introduced or otherwise provided. According to this aspect, the guide RNA and the enzymatically active Cas9 optionally having a functional group attached thereto are nonnaturally occurring in the cell in which they are introduced, or otherwise provided. To this extent, cells may be genetically engineered or genetically modified to include the CRISPR /Cas systems described herein.

One such CRISPR/Cas system uses the S. pyogenes Cas9 nuclease (Sp. Cas9), an extremely high-affinity (see Sternberg, S. H., Redding, S., Jinek, M., Greene, E. C. & Doudna, J. A. DNA interrogation by the CRISPR RNA-guided endonuclease Cas9. Nature 507, 62-67 (2014) hereby incorporated by reference in its entirety), programmable DNA-binding protein isolated from a type II CRISPR-associated system (see Garneau, J. E. et al. The CRISPR/Cas bacterial immune system cleaves bacteriophage and plasmid DNA. Nature 468, 67-71 (2010) and Jinek, M. et al. A programmable dual-RNA-guided DNA endonuclease in adaptive bacterial immunity. Science 337, 816-821 (2012) each of which are hereby incorporated by reference in its entirety). The DNA locus targeted by Cas9 precedes a three nucleotide (nt) 5′-NGG-3′ “PAM” sequence, and matches a 15-22-nt guide or spacer sequence within a Cas9-bound RNA cofactor, referred to herein and in the art as a guide RNA. Altering this guide RNA is sufficient to target Cas9 to a target nucleic acid. In a multitude of CRISPR-based biotechnology applications, the guide is often presented in a so-called sgRNA (single guide RNA), wherein the two natural Cas9 RNA cofactors (gRNA and tracrRNA) are fused via an engineered loop.

Embodiments of the present disclosure are directed to a method of delivering a functional group or moiety attached to a enzymatically active Cas9 protein to a target nucleic acid in a cell comprising providing to the cell the enzymatically active Cas9 protein having the functional group or moiety attached thereto and a guide RNA having spacer sequence between 14 and 8 nucleotides in length wherein the guide RNA and the Cas9 protein form a co-localization complex with the target nucleic acid and where the enzymatically active Cas9 protein is inactivated and where the functional group or moiety is delivered to the target nucleic acid. Methods described herein can be performed in vitro, in vivo or ex vivo. According to one aspect, the cell is a eukaryotic cell or a prokaryotic cell. According to one aspect, the cell is a bacteria cell, a yeast cell, a mammalian cell, a plant cell or an animal cell. According to one aspect, the Cas9 protein is an enzymatically active Cas9 protein, a Cas9 protein wild-type protein, or a enzymatically active Cas9 nickase. Additional exemplary Cas9 proteins include Cas9 proteins attached to, bound to or fused with functional proteins such as transcriptional regulators, such as transcriptional activators or repressors, a Fok-domain, such as Fok 1, an aptamer, a binding protein, PP7, MS2 and the like.

According to certain aspects, the enzymatically active Cas9 protein having the functional group attached thereto may be delivered directly to a cell as a native species by methods known to those of skill in the art, including injection or lipofection, or as translated from its cognate mRNA, or transcribed from its cognate DNA into mRNA (and thereafter translated into protein). Cas9 DNA and mRNA may be themselves introduced into cells through electroporation, transient and stable transfection (including lipofection) and viral transduction or other methods known to those of skill in the art. According to certain aspects, the guide RNA may be delivered directly to a cell as a native species by methods known to those of skill in the art, including injection or lipofection, or as transcribed from its cognate DNA, with the cognate DNA introduced into cells through electroporation, transient and stable transfection (including lipofection) and viral transduction.

According to certain aspects, a first nucleic acid encoding an enzymatically active Cas9 optionally having a functional group attached thereto is provided to a cell. A second nucleic acid encoding guide RNA complementary to the target nucleic acid is provided to the cell. The cell expresses the guide RNA and the enzymatically active Cas9 optionally having a functional group attached thereto, wherein the guide RNA and the Cas9 protein form a co-localization complex with the target nucleic acid thereby delivering the functional group, if present, to the target nucleic acid. According to one aspect, the guide RNA is an enzymatic guide RNA and the enzymatically active Cas9 optionally having a functional group attached thereto cuts or nicks the target nucleic acid. According to one aspect, the guide RNA is an nonenzymatic guide RNA and the enzymatically active Cas9 optionally having a functional group attached thereto acts as a nuclease null Cas9 and delivers the functional group, if present, to the target nucleic acid where the functional group performs the function of the functional group. According to one aspect, the first nucleic acid encoding the Cas9 protein and the second nucleic acid encoding the guide RNA may be present on the same or different vectors. The cell may be any desired cell including a eukaryotic cell. An exemplary cell is a human cell. An exemplary cell is a stem cell, whether adult or embryonic. An exemplary cell is an induced pluripotent stem cell. An exemplary cell is an embryonic stem cell. According to this aspect, the embryonic stem cell which may then be implanted into an animal where the embryonic stem cell differentiates into a particular desired tissue type and the tissue type expresses the nucleic acids encoding the Cas9 and the guide RNA.

Embodiments of the present disclosure are directed to a method of delivering an enzymatically active Cas9 protein optionally having a functional group attached thereto to cells within a subject comprising administering to the subject, such as systemically administering to the subject, such as by intravenous administration or injection, intraperitoneal administration or injection, intramuscular administration or injection, intracranial administration or injection, intraocular administration or injection, subcutaneous administration or injection, a enzymatically active Cas9 protein optionally having a functional group attached thereto or a nucleic acid encoding the enzymatically active Cas9 protein optionally having a functional group attached thereto.

Embodiments of the present disclosure are directed to a method of delivering a guide RNA to cells within a subject comprising administering to the subject, such as systemically administering to the subject, such as by intravenous administration or injection, intraperitoneal administration or injection, intramuscular administration or injection, intracranial administration or injection, intraocular administration or injection, subcutaneous administration or injection, a guide RNA or a nucleic acid encoding the guide RNA.

Embodiments of the present disclosure are directed to a method of delivering an enzymatically active Cas9 protein optionally having a functional group attached thereto and a guide RNA to cells within a subject comprising administering to the subject, such as systemically administering to the subject, such as by intravenous administration or injection, intraperitoneal administration or injection, intramuscular administration or injection, intracranial administration or injection, intraocular administration or injection, subcutaneous administration or injection, an enzymatically active Cas9 optionally having a functional group attached thereto or a nucleic acid encoding the enzymatically active Cas9 protein optionally having a functional group attached thereto and a guide RNA or a nucleic acid encoding the guide RNA.

Cas9 proteins and Type II CRISPR systems are well documented in the art. See Makarova et al., Nature Reviews, Microbiology, Vol. 9, June 2011, pp. 467-477 including all supplementary information hereby incorporated by reference in its entirety. In general, bacterial and archaeal CRISPR-Cas systems rely on short guide RNAs in complex with Cas proteins to direct degradation of complementary sequences present within invading foreign nucleic acid. See Deltcheva, E. et al. CRISPR RNA maturation by trans-encoded small RNA and host factor RNase III. Nature 471, 602-607 (2011); Gasiunas, G., Barrangou, R., Horvath, P. & Siksnys, V. Cas9-crRNA ribonucleoprotein complex mediates specific DNA cleavage for adaptive immunity in bacteria. Proceedings of the National Academy of Sciences of the United States of America 109, E2579-2586 (2012); Jinek, M. et al. A programmable dual-RNA-guided DNA endonuclease in adaptive bacterial immunity. Science 337, 816-821 (2012); Sapranauskas, R. et al. The Streptococcus thermophilus CRISPR/Cas system provides immunity in Escherichia coli. Nucleic acids research 39, 9275-9282 (2011); and Bhaya, D., Davison, M. & Barrangou, R. CRISPR-Cas systems in bacteria and archaea: versatile small RNAs for adaptive defense and regulation. Annual review of genetics 45, 273-297 (2011). A recent in vitro reconstitution of the S. pyogenes type II CRISPR system demonstrated that crRNA (“CRISPR RNA”) fused to a normally trans-encoded tracrRNA (“trans-activating CRISPR RNA”) is sufficient to direct Cas9 protein to sequence-specifically cleave target DNA sequences matching the crRNA. Expressing a gRNA homologous to a target site results in Cas9 recruitment and degradation of the target DNA. See H. Deveau et al., Phage response to CRISPR-encoded resistance in Streptococcus thermophilus. Journal of Bacteriology 190, 1390 (Feb, 2008).

Three classes of CRISPR systems are generally known and are referred to as Type I, Type II or Type III). According to one aspect, a particular useful enzyme according to the present disclosure to cleave dsDNA is the single effector enzyme, Cas9, common to Type II. See K. S. Makarova et al., Evolution and classification of the CRISPR-Cas systems. Nature reviews. Microbiology 9, 467 (Jun, 2011) hereby incorporated by reference in its entirety. Within bacteria, the Type II effector system consists of a long pre-crRNA transcribed from the spacer-containing CRISPR locus, the multifunctional Cas9 protein, and a tracrRNA important for gRNA processing. The tracrRNAs hybridize to the repeat regions separating the spacers of the pre-crRNA, initiating dsRNA cleavage by endogenous RNase III, which is followed by a second cleavage event within each spacer by Cas9, producing mature crRNAs that remain associated with the tracrRNA and Cas9. TracrRNA-crRNA fusions are contemplated for use in the present methods.

According to one aspect, the enzyme of the present disclosure, such as Cas9 unwinds the DNA duplex and searches for sequences matching the crRNA to cleave. Target recognition occurs upon detection of complementarity between a “protospacer” sequence in the target DNA and the remaining spacer sequence in the crRNA. Importantly, Cas9 cuts the DNA only if a correct protospacer-adjacent motif (PAM) is also present at the 3′ end. According to certain aspects, different protospacer-adjacent motif can be utilized. For example, the S. pyogenes system requires an NGG sequence, where N can be any nucleotide. S. thermophilus Type II systems require NGGNG (see P. Horvath, R. Barrangou, CRISPR/Cas, the immune system of bacteria and archaea. Science 327, 167 (Jan. 8, 2010) hereby incorporated by reference in its entirety and NNAGAAW (see H. Deveau et al., Phage response to CRISPR-encoded resistance in Streptococcus thermophilus. Journal of bacteriology 190, 1390 (February, 2008) hereby incorporatd by reference in its entirety), respectively, while different S. mutans systems tolerate NGG or NAAR (see J. R. van der Ploeg, Analysis of CRISPR in Streptococcus mutans suggests frequent occurrence of acquired immunity against infection by M102-like bacteriophages. Microbiology 155, 1966 (June, 2009) hereby incorporated by reference in its entirety. Bioinformatic analyses have generated extensive databases of CRISPR loci in a variety of bacteria that may serve to identify additional useful PAMs and expand the set of CRISPR-targetable sequences (see M. Rho, Y. W. Wu, H. Tang, T. G. Doak, Y. Ye, Diverse CRISPRs evolving in human microbiomes. PLoS genetics 8, e1002441 (2012) and D. T. Pride et al., Analysis of streptococcal CRISPRs from human saliva reveals substantial sequence diversity within and between subjects over time. Genome research 21, 126 (January, 2011) each of which are hereby incorporated by reference in their entireties.

In S. pyogenes, Cas9 generates a blunt-ended double-stranded break 3 bp upstream of the protospacer-adjacent motif (PAM) via a process mediated by two catalytic domains in the protein: an HNH domain that cleaves the complementary strand of the DNA and a RuvC-like domain that cleaves the non-complementary strand. See Jinek et al., Science 337, 816-821 (2012) hereby incorporated by reference in its entirety. Cas9 proteins are known to exist in many Type II CRISPR systems including the following as identified in the supplementary information to Makarova et al., Nature Reviews, Microbiology, Vol. 9, June 2011, pp. 467-477: Methanococcus maripaludis C7; Corynebacterium diphtheriae; Corynebacterium efficiens YS-314; Corynebacterium glutamicum ATCC 13032 Kitasato; Corynebacterium glutamicum ATCC 13032 Bielefeld; Corynebacterium glutamicum R; Corynebacterium kroppenstedtii DSM 44385; Mycobacterium abscessus ATCC 19977; Nocardia farcinica IFM10152; Rhodococcus erythropolis PR4; Rhodococcus jostii RHA 1; Rhodococcus opacus B4 uid36573; Acidothermus cellulolyticus 11B; Arthrobacter chlorophenolicus A6; Kribbella flavida DSM 17836 uid43465; Thermomonospora curvata DSM 43183; Bifidobacterium dentium Bd1; Bifidobacterium longum DJ010A; Slackia heliotrinireducens DSM 20476; Persephonella marina EX H1; Bacteroides fragilis NCTC 9434; Capnocytophaga ochracea DSM 7271; Flavobacterium psychrophilum JIP02 86; Akkermansia muciniphila ATCC BAA 835; Roseiflexus castenholzii DSM 13941; Roseiflexus RS1; Synechocystis PCC6803; Elusimicrobium minutum Pei191; uncultured Termite group 1 bacterium phylotype Rs D17; Fibrobacter succinogenes S85; Bacillus cereus ATCC 10987; Listeria innocua; Lactobacillus casei; Lactobacillus rhamnosus GG; Lactobacillus salivarius UCC118; Streptococcus agalactiae A909; Streptococcus agalactiae NEM316; Streptococcus agalactiae 2603; Streptococcus dysgalactiae equisimilis GGS 124; Streptococcus equi zooepidemicus MGCS10565; Streptococcus gallolyticus UCN34 uid46061; Streptococcus gordonii Challis subst CH1; Streptococcus mutans NN2025 uid46353; Streptococcus mutans; Streptococcus pyogenes M1 GAS; Streptococcus pyogenes MGAS5005; Streptococcus pyogenes MGAS2096; Streptococcus pyogenes MGAS9429; Streptococcus pyogenes MGAS10270; Streptococcus pyogenes MGAS6180; Streptococcus pyogenes MGAS315; Streptococcus pyogenes SSI-1; Streptococcus pyogenes MGAS10750; Streptococcus pyogenes NZ131; Streptococcus thermophiles CNRZ1066; Streptococcus thermophiles LMD-9; Streptococcus thermophiles LMG 18311; Clostridium botulinum A3 Loch Maree; Clostridium botulinum B Eklund 17B; Clostridium botulinum Ba4 657; Clostridium botulinum F Langeland; Clostridium cellulolyticum H10; Finegoldia magna ATCC 29328; Eubacterium rectale ATCC 33656; Mycoplasma gallisepticum; Mycoplasma mobile 163K; Mycoplasma penetrans; Mycoplasma synoviae 53; Streptobacillus moniliformis DSM 12112; Bradyrhizobium BTAil; Nitrobacter hamburgensis X14; Rhodopseudomonas palustris BisB18; Rhodopseudomonas palustris BisB5; Parvibaculum lavamentivorans DS-1; Dinoroseobacter shibae DFL 12; Gluconacetobacter diazotrophicus Pal 5 FAPERJ; Gluconacetobacter diazotrophicus Pal 5 JGI; Azospirillum B510 uid46085; Rhodospirillum rubrum ATCC 11170; Diaphorobacter TPSY uid29975; Verminephrobacter eiseniae EF01-2; Neisseria meningitides 053442; Neisseria meningitides alpha14; Neisseria meningitides Z2491; Desulfovibrio salexigens DSM 2638; Campylobacter jejuni doylei 269 97; Campylobacter jejuni 81116; Campylobacter jejuni; Campylobacter lari RM2100; Helicobacter hepaticus; Wolinella succinogenes; Tolumonas auensis DSM 9187; Pseudoalteromonas atlantica T6c; Shewanella pealeana ATCC 700345; Legionella pneumophila Paris; Actinobacillus succinogenes 130Z; Pasteurella multocida; Francisella tularensis novicida U112; Francisella tularensis holarctica; Francisella tularensis FSC 198; Francisella tularensis tularensis; Francisella tularensis WY96-3418; and Treponema denticola ATCC 35405. The Cas9 protein may be referred by one of skill in the art in the literature as Csn1. An exemplary S. pyogenes Cas9 protein sequence is shown below. See Deltcheva et al., Nature 471, 602-607 (2011) hereby incorporated by reference in its entirety.

MDKKYSIGLDIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEV
QTGGFSKESILPKRNSDKLIARKKDWDPKKYGGFDSPTVAYSVLVVAKVE
KGKSKKLKSVKELLGITIMERSSFEKNPIDFLEAKGYKEVKKDLIIKLPK
YSLFELENGRKRMLASAGELQKGNELALPSKYVNFLYLASHYEKLKGSPE
DNEQKQLFVEQHKHYLDEIIEQISEFSKRVILADANLDKVLSAYNKHRDK
PIREQAENIIHLFTLTNLGAPAAFKYFDTTIDRKRYTSTKEVLDATLIHQ
SITGLYETRIDLSQLGGD.

Modification to the Cas9 protein is a representative embodiment of the present disclosure. CRISPR systems useful in the present disclosure are described in R. Barrangou, P. Horvath, CRISPR: new horizons in phage resistance and strain identification. Annual review of food science and technology 3, 143 (2012) and B. Wiedenheft, S. H. Sternberg, J. A. Doudna, RNA-guided genetic silencing systems in bacteria and archaea. Nature 482, 331 (Feb. 16, 2012) each of which are hereby incorporated by reference in their entireties.

According to one aspect, a Cas9 protein having two or more nuclease domains may be modified or altered to inactivate all but one of the nuclease domains. Such a modified or altered Cas9 protein is referred to as a nickase, to the extent that the nickase cuts or nicks only one strand of double stranded DNA. According to one aspect, the Cas9 protein or Cas9 protein nickase includes homologs and orthologs thereof which retain the ability of the protein to bind to the DNA and be guided by the RNA. According to one aspect, the Cas9 protein includes the sequence as set forth for naturally occurring Cas9 from S. pyogenes and protein sequences having at least 30%, 40%, 50%, 60%, 70%, 80%, 90%, 95%, 98% or 99% homology thereto and being a DNA binding protein, such as an RNA guided DNA binding protein.

Target nucleic acids include any nucleic acid sequence to which a co-localization complex as described herein can be useful to either cut, nick, regulate, identify, influence or otherwise target for other useful purposes using the methods described herein. Target nucleic acids include cellular RNA. Target nucleic acids include cellular DNA. Target nucleic acids include genes. For purposes of the present disclosure, DNA, such as double stranded DNA, can include the target nucleic acid and a co-localization complex can bind to or otherwise co-localize with the DNA at or adjacent or near the target nucleic acid and in a manner in which the co-localization complex may have a desired effect on the target nucleic acid. Such target nucleic acids can include endogenous (or naturally occurring) nucleic acids and exogenous (or foreign) nucleic acids. One of skill based on the present disclosure will readily be able to identify or design guide RNAs and Cas9 proteins which co-localize to a DNA including a target nucleic acid. One of skill will further be able to identify transcriptional regulator proteins or domains which likewise co-localize to a DNA including a target nucleic acid. DNA includes genomic DNA, mitochondrial DNA, viral DNA or exogenous DNA.

Foreign nucleic acids (i.e. those which are not part of a cell's natural nucleic acid composition) may be introduced into a cell using any method known to those skilled in the art for such introduction. Such methods include transfection, transduction, viral transduction, microinjection, lipofection, nucleofection, nanoparticle bombardment, transformation, conjugation and the like. One of skill in the art will readily understand and adapt such methods using readily identifiable literature sources.

Vectors are contemplated for use with the methods and constructs described herein. The term “vector” includes a nucleic acid molecule capable of transporting another nucleic acid to which it has been linked. Vectors used to deliver the nucleic acids to cells as described herein include vectors known to those of skill in the art and used for such purposes. Certain exemplary vectors may be plasmids, lentiviruses or adeno-associated viruses known to those of skill in the art. Vectors include, but are not limited to, nucleic acid molecules that are single-stranded, doublestranded, or partially double-stranded; nucleic acid molecules that comprise one or more free ends, no free ends (e.g. circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g. retroviruses, lentiviruses, replication defective retroviruses, adenoviruses, replication defective adenoviruses, and adeno-associated viruses). Viral vectors also include polynucleotides carried by a virus for transfection into a host cell. Certain vectors are capable of autonomous replication in a host cell into which they are introduced (e.g. bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors.” Common expression vectors of utility in recombinant DNA techniques are often in the form of plasmids. Recombinant expression vectors can comprise a nucleic acid of the invention in a form suitable for expression of the nucleic acid in a host cell, which means that the recombinant expression vectors include one or more regulatory elements, which may be selected on the basis of the host cells to be used for expression, that is operatively-linked to the nucleic acid sequence to be expressed. Within a recombinant expression vector, “operably linked” or “operatively linked” is intended to mean that the nucleotide sequence of interest is linked to the regulatory element(s) in a manner that allows for expression of the nucleotide sequence (e.g. in an in vitro transcription/translation system or in a host cell when the vector is introduced into the host cell).

Methods of non-viral delivery of nucleic acids or native DNA binding protein, native guide RNA or other native species include lipofection, microinjection, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Felgner, WO 91/17424; WO 91/16024. Delivery can be to cells (e.g. in vitro or ex vivo administration) or target tissues (e.g. in vivo administration). The term native includes the protein, enzyme or guide RNA species itself and not the nucleic acid encoding the species.

Regulatory elements are contemplated for use with the methods and constructs described herein. The term “regulatory element” is intended to include promoters, enhancers, internal ribosomal entry sites (IRES), and other expression control elements (e.g. transcription termination signals, such as polyadenylation signals and poly-U sequences). Such regulatory elements are described, for example, in Goeddel, GENE EXPRESSION TECHNOLOGY: METHODS IN ENZYMOLOGY 185, Academic Press, San Diego, Calif. (1990). Regulatory elements include those that direct constitutive expression of a nucleotide sequence in many types of host cell and those that direct expression of the nucleotide sequence only in certain host cells (e.g., tissue-specific regulatory sequences). A tissue-specific promoter may direct expression primarily in a desired tissue of interest, such as muscle, neuron, bone, skin, blood, specific organs (e.g. liver, pancreas), or particular cell types (e.g. lymphocytes). Regulatory elements may also direct expression in a temporal-dependent manner, such as in a cell-cycle dependent or developmental stage-dependent manner, which may or may not also be tissue or cell-type specific. In some embodiments, a vector may comprise one or more pol III promoter (e.g. 1, 2, 3, 4, 5, or more pol III promoters), one or more pol II promoters (e.g. 1, 2, 3, 4, 5, or more pol II promoters), one or more pol I promoters (e.g. 1, 2, 3, 4, 5, or more pol I promoters), or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer) [see, e.g., Boshart et al, Cell, 41:521-530 (1985)], the SV40 promoter, the dihydrofolate reductase promoter, the β-actin promoter, the phosphoglycerol kinase (PGK) promoter, and the EF1α promoter and Pol II promoters described herein. Also encompassed by the term “regulatory element” are enhancer elements, such as WPRE; CMV enhancers; the R-U5′ segment in LTR of HTLV-I (Mol. Cell. Biol., Vol. 8(1), p. 466-472, 1988); SV40 enhancer; and the intron sequence between exons 2 and 3 of rabbit β-globin (Proc. Natl. Acad. Sci. USA., Vol. 78(3), p. 1527-31, 1981). It will be appreciated by those skilled in the art that the design of the expression vector can depend on such factors as the choice of the host cell to be transformed, the level of expression desired, etc. A vector can be introduced into host cells to thereby produce transcripts, proteins, or peptides, including fusion proteins or peptides, encoded by nucleic acids as described herein (e.g., clustered regularly interspersed short palindromic repeats (CRISPR) transcripts, proteins, enzymes, mutant forms thereof, fusion proteins thereof, etc.).

Aspects of the methods described herein may make use of terminator sequences. A terminator sequence includes a section of nucleic acid sequence that marks the end of a gene or operon in genomic DNA during transcription. This sequence mediates transcriptional termination by providing signals in the newly synthesized mRNA that trigger processes which release the mRNA from the transcriptional complex. These processes include the direct interaction of the mRNA secondary structure with the complex and/or the indirect activities of recruited termination factors. Release of the transcriptional complex frees RNA polymerase and related transcriptional machinery to begin transcription of new mRNAs. Terminator sequences include those known in the art and identified and described herein.

Aspects of the methods described herein may make use of epitope tags and reporter gene sequences. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporter genes include, but are not limited to, glutathione-S-transferase (GST), horseradish peroxidase (HRP), chloramphenicol acetyltransferase (CAT) beta-galactosidase, betaglucuronidase, luciferase, green fluorescent protein (GFP), HcRed, DsRed, cyan fluorescent protein (CFP), yellow fluorescent protein (YFP), and autofluorescent proteins including blue fluorescent protein (BFP).

The following examples are set forth as being representative of the present disclosure. These examples are not to be construed as limiting the scope of the present disclosure as these and other equivalent embodiments will be apparent in view of the present disclosure, figures and accompanying claims.

EXAMPLE I

### Fluorescent Reporter Assay for Quantifying Cas9 Deletions

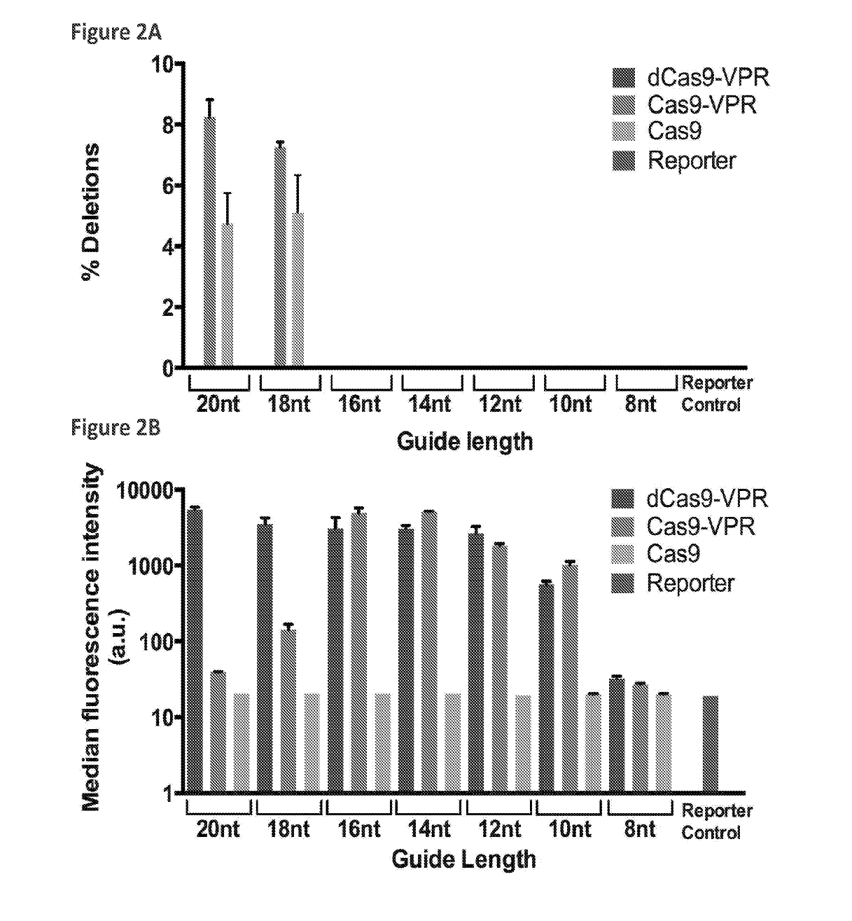

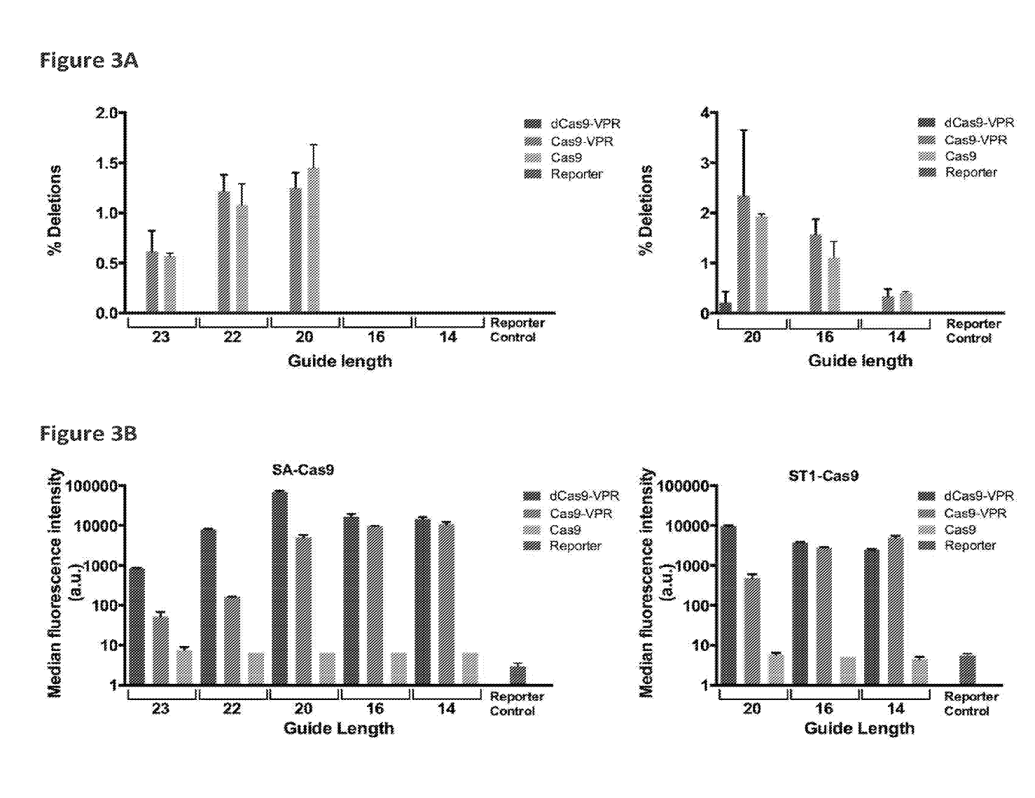

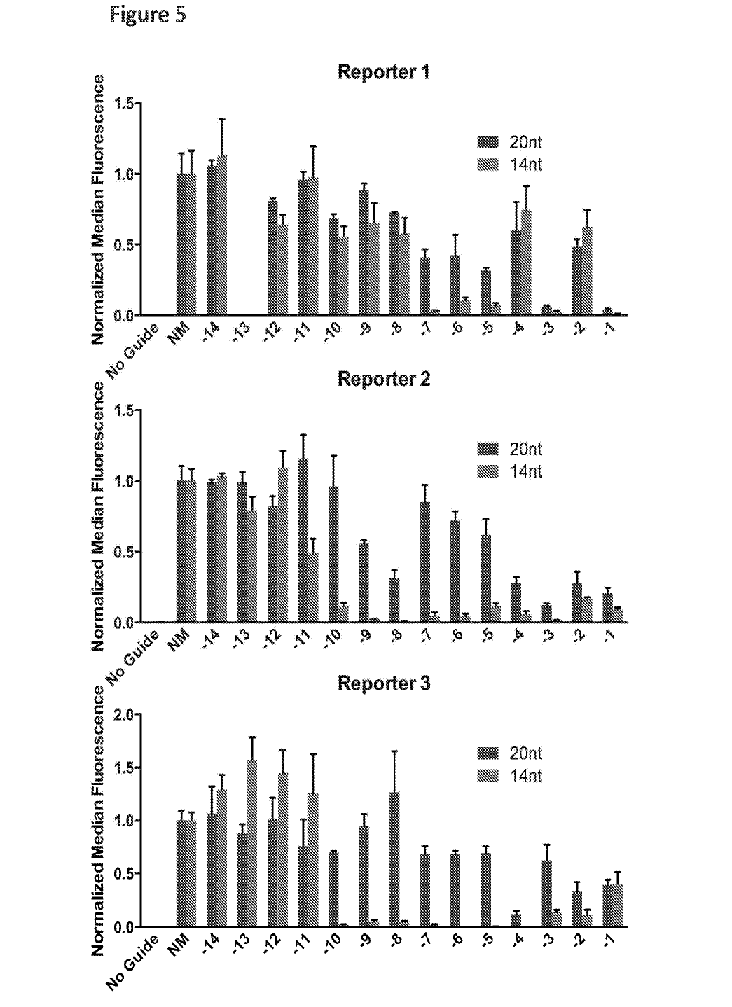

Fluorescent reporter experiments the results of which are shown in FIGS. 2A and 2B and FIGS. 3A and 3B were conducted with an Addgene #47320 plasmid modified to include an extra gRNA binding site 100 bp upstream of the already existing one. For ST1 and SA Cas9 experiments, the protospacer remained the same but the PAM sequence was modified as needed for ST1 or SA Cas9. For FIG. 5, all experiments were conducted with a reporter with a single gRNA binding site. Reporter 1 denotes Addgene #47320, reporters 2 and 3 are similar to reporter 1 except the protospacer and PAM (in bold) were changed to contain the sequence GGGGCCACTAGGGACAGGATTGG and AAGAGAGACAGTACATGCCCTGG respectively. gRNAs of various spacer lengths were co-transfected along with the indicated Cas9 protein and reporter into HEK293T cells along with an EBFP2 transfection control. Cells were analyzed by flow cytometry 48 hours post transfection and then when necessary were lysed to extract genomic DNA.

### EXAMPLE II

### Reporter Deletion Analysis

DNA was extracted using QuickExtract DNA Extraction Solution (Epicentre). DNA was then used for PCR to amplify desired regions. The amplified samples were then run on a 2% agarose gel stained with GelGreen (Biotium) and visualized using Gel Doc EZ (Bio-Rad). Band intensity was quantified using GelAnalyzer.

### EXAMPLE III

### qRT-PCR Analysis

Samples were lysed and RNA was extracted using the RNeasy Plus Mini Kit (Qiagen). cDNA was made using the iScript cDNA synthesis kit (Bio-Rad) with 500 ng of RNA. KAPA SYBR FAST Universal 2× qPCR Master Mix (Kapa Biosystems) was used for qPCR with 0.5 μl of cDNA used for each reaction. Activation was analyzed using CFX96 Real-Time PCR Detection System (Bio-Rad). Gene expression levels were normalized to β-actin levels.

### EXAMPLE IV

### Endogenous Indel Analysis

DNA was extracted from 24-well plates using 350 μl of QuickExtract DNA Extraction Solution (Epicentre), according to the manufacturing instructions. Amplicon library preparation was performed using two PCRs. The first PCR amplified from the genome adding appropriate barcodes and parts of adapters for Illumina sequencing. The second PCR extended out the Illumina adapters. In the first PCR, 5 uL of extracted DNA was used as template in a 100 uL Kapa HiFi PCR reaction and run for 30 cycles. PCR products were then purified using a homemade SPRI bead mixture and eluted in 50 uL of elution buffer. For the second PCR, 2 uL of the previous first round PCR was used as template in a 25 uL reaction and PCRs were run for a total of 9 cycles. PCR products were then run on an agarose gel, extracted and column purified. Equal amounts of each sample were then pooled and sequenced on an Illumina MiSeq using the paired end 150 MiSeq Nano kit.

Mate pair reads were merged into single contigs using FLASH. See T. Magoc and S. L. Salzberg, Bioinformatics 27 (21), 2957 (2011). Each contig was then mapped to a custom reference representing the three amplicons using bwa mem. See H. Li and R. Durbin, Bioinformatics 25 (14), 1754 (2009). SAM output files were then converted to BAM files and pileup files were generated for each sample using SAM tools. See H. Li, B. Handsaker, A. Wysoker et al., Bioinformatics 25 (16), 2078 (2009). Pileup files were then analyzed using custom python scripts to determine observed mutation rates. Mutations were only counted if the mutations spanned some portion of the sgRNA target site. In addition, base quality scores of ≥28 were also required for any mutations to be called. To minimize the impact of sequencing error, single base substitutions were excluded in this analysis.

### EXAMPLE V

### RNA Sequencing for Quantifying Activator Specificity

For each sample, 200 ng of total RNA was polyA selected using Dynabeads mRNA Purification Kit (Life Technologies). The RNA was then DNAse treated with Turbo DNase (Life Technologies) and cleaned up with Agenocourt RNAClean XP Beads (Beckman Coulter). RNA-Seq Libraries were made using the NEBNext Ultra RNA Library Prep Kit for Illumina (New England BioLabs) according to manufacturer's instructions with NEBNext Multiplex Oligos (New England BioLabs). Libraries were analyzed on a BioAnalyzer using a High Sensitivity DNA Analysis Kit (Agilent). Libraries were then quantified using a KAPA Library Quantification Kit (KAPA Biosystems) and pooled to a final concentration of 4 nM. Sequencing was performed on an Illumina NextSeq instrument with paired end reads. Reads were aligned to the hg19 UCSC Known Genes annotations using RSEM v1.2.1 and analyzed in Python and R. Differential gene expression analysis was done using the Voom and Limma packages in R for all genes with ≥1 TPM in each replicate, and a one-way within-subjects ANOVA was performed on the number of differentially expressed genes for each condition to quantify off-target effects, where differential expression was defined by Benjamini-Hochberg adjusted p-value <0.05 and fold-change >2 or <0.5.

### EXAMPLE VI

### Cell Culture for Endogenous Target Mutation/Activation or Deletion Reporter

HEK-293T cells were cultivated in Dulbecco' s Modified Eagle Medium (Life Technologies) with 10% FBS (Life Technologies) and Penicillin/Streptomycin (Life Technologies). Incubator conditions were 37° C. and 5% CO₂. Cells were tested for mycoplasma yearly. Cells were seeded into 24-well plates at 50,000 cells per well and transfected with 200 ng of Cas9 construct, lOng of guide and 60 ng of reporter (when necessary) via Lipofectamine 2000 (Life Technologies). Post transfection, cells were grown for 48-72 hours and lysed for either RNA or DNA extraction.

### EXAMPLE VII

### Vector Design and Construction

Reporter gRNA was previously described (Addgene #48672), dCas9-VPR was previously described (Addgene #63798) and Cas9 was described (Addgene#41815). Cas9-VPR was cloned via Gateway assembly (Invitrogen) based on the Cas9 plasmid. gRNAs for endogenous targets were cloned into Addgene #41817 and transiently transfected.

### EXAMPLE VIII

### Determining Effect of Progressive gRNA Truncation on Cas9 Nuclease Activity

The effect of progressive gRNA truncation on enzymatically active Cas9 nuclease activity was determined. Cas9 was targeted along with a set of truncated gRNAs to the promoter of a transiently transfected fluorescent reporter. Cas9 showed robust levels of nuclease activity, i.e. cutting of the promoter, with either 20 or 18 nt gRNAs and sharp loss of nuclease function when 16 nt or shorter gRNAs were used. See FIG. 2A. To determine if the lack of observed DNA modification for ≤16 nt guides was due to attenuated Cas9 nuclease activity, a potent activator (VPR) was fused to Cas9 according to A. Chavez, J. Scheiman, S. Vora et al., Nature methods 12 (4), 326 (2015). The Cas9-VPR fusion was then targeted to the same fluorescent reporter and the effect of gRNA length on activation was quantified. Cas9-VPR showed minimal activation when a 20 nt gRNA was used, but when the gRNA length was decreased, a corresponding increase in activation was observed, achieving maximal activation with 16 or 14 nt gRNAs. See FIG. 2B. When compared to wild-type Cas9, Cas9-VPR showed similar nuclease activity with 20 or 18 nt gRNAs. A fusion between nuclease null Cas9 and VPR (dCas9-VPR) showed an equivalent reporter activation compared to Cas9-VPR when 16 or 14 nt gRNAs were employed. See FIGS. 2A and 2B.

To assess the generality of this approach, we tested the effects of shortened guide RNAs on nuclease activity was tested with two other VPR-fused Cas9 proteins, Staphylococcus aureus Cas9 (SA-Cas9) and Streptococcus thermophilus Cas9 (ST1-Cas9). See K. M. Esvelt, P. Mali, J. L. Braff et al., Nature methods 10 (11), 1116 (2013) and F. A. Ran, L. Cong, W. X. Yan et al., Nature 520 (7546), 186 (2015). As with SP-Cas9, a similar capacity of shortened guide RNAs to inhibit nuclease activity, while still allowing SA and ST1-Cas9 to interact with DNA was observed (See FIGS. 3A and 3B).

Accordingly, the length of the spacer sequence of the guide RNA dictated whether the Cas9-VPR fusion cut the target nucleic acid or whether the target gene was activated by the transcriptional activator.

### EXAMPLE IX

### Determining Effect of Progressive gRNA Truncation on Cas9 Nuclease Activity

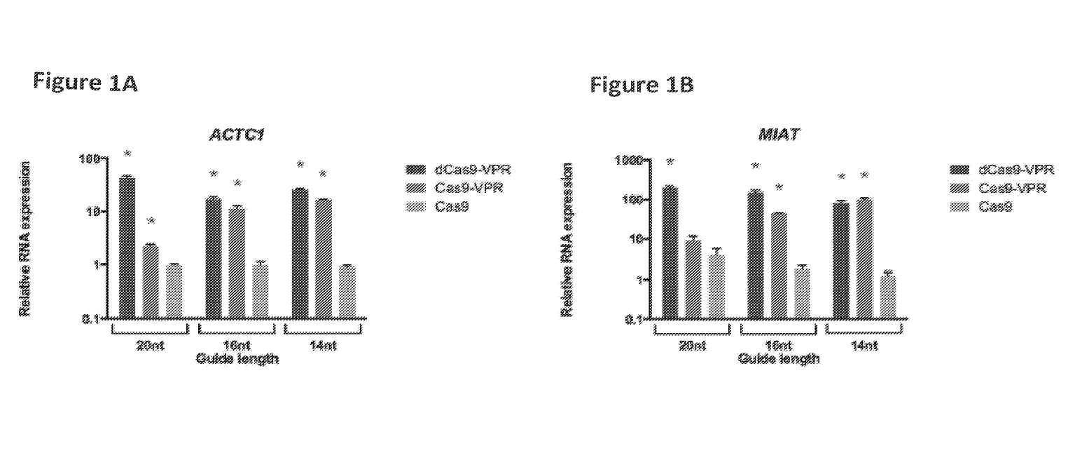

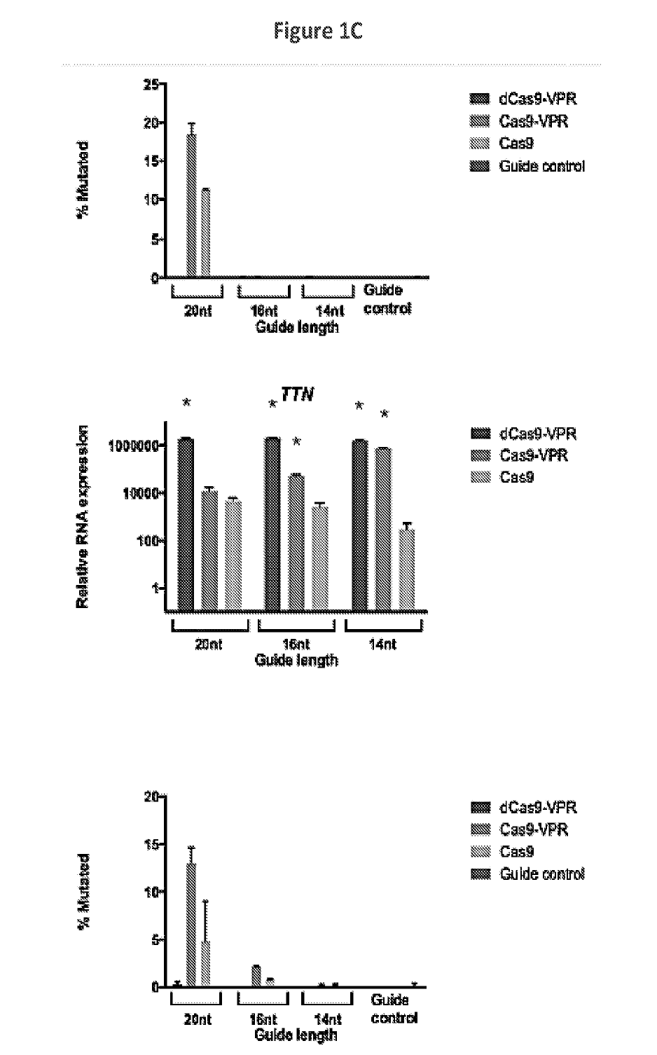

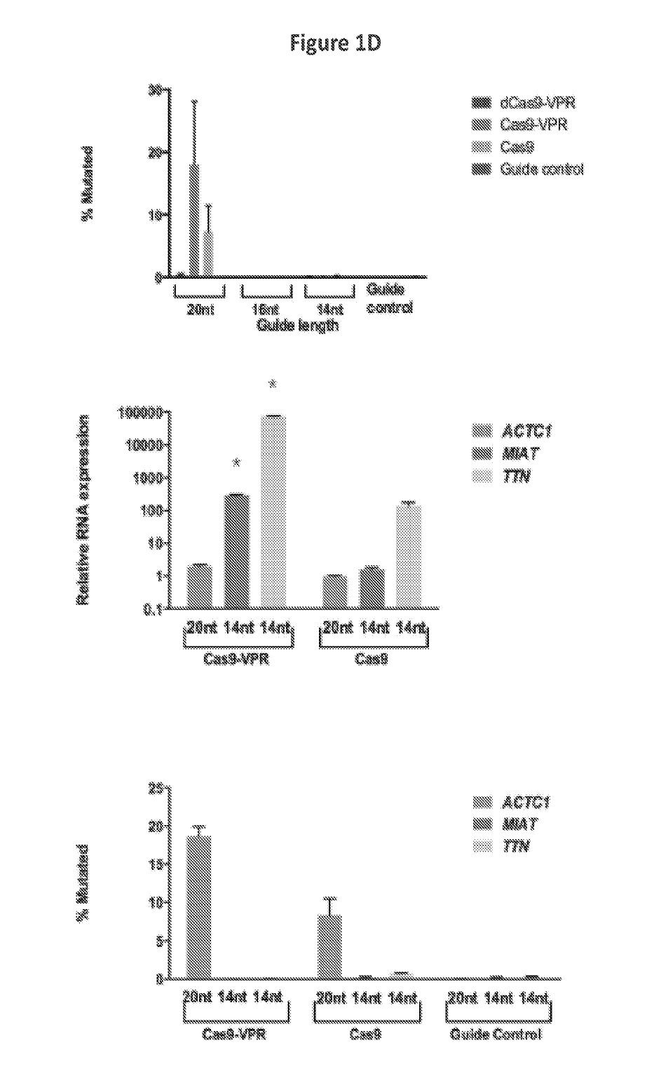

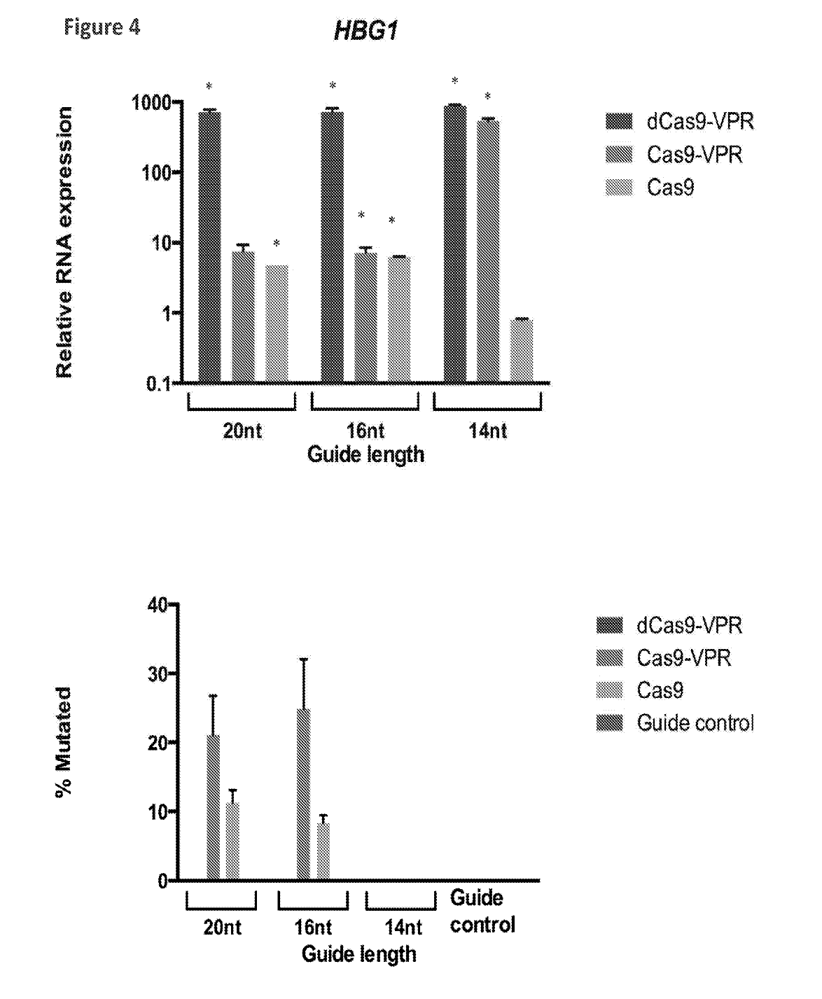

The effect of gRNA spacer sequence length on Cas9 activity at endogenous target genes was determined. Using 20, 16, or 14 nt gRNAs, Cas9, Cas9-VPR, or dCas9-VPR was targeted to the promoter region of a set of structural coding genes (ACTC1 and TTN) and a long noncoding RNA (MIAT) and a protein critical to tissue oxygen delivery (HBG1). Similar to the transiently transfected fluorescent reporter, Cas9-VPR was able to induce target chromosomal gene expression with 16 or 14 nt, but not 20 nt gRNAs. See FIGS. 1A-1C and FIG. 4. Cas9-VPR in conjunction with a 14 nt gRNA was able to generate at least 40% of the level of expression for all targets tested when compared to dCas9-VPR with a 20 nt gRNA. Along with measuring gene induction, the amount of Cas9 induced indels within targeted promoter regions was examined. For ACTC1 and MIAT, Cas9-dependent mutagenesis was only observed with 20 nt gRNAs. See FIGS. 1A-1B. For TTN and HBG1, indels were observed with both 20 and 16 nt gRNAs. See FIG. 1C and FIG. 4.

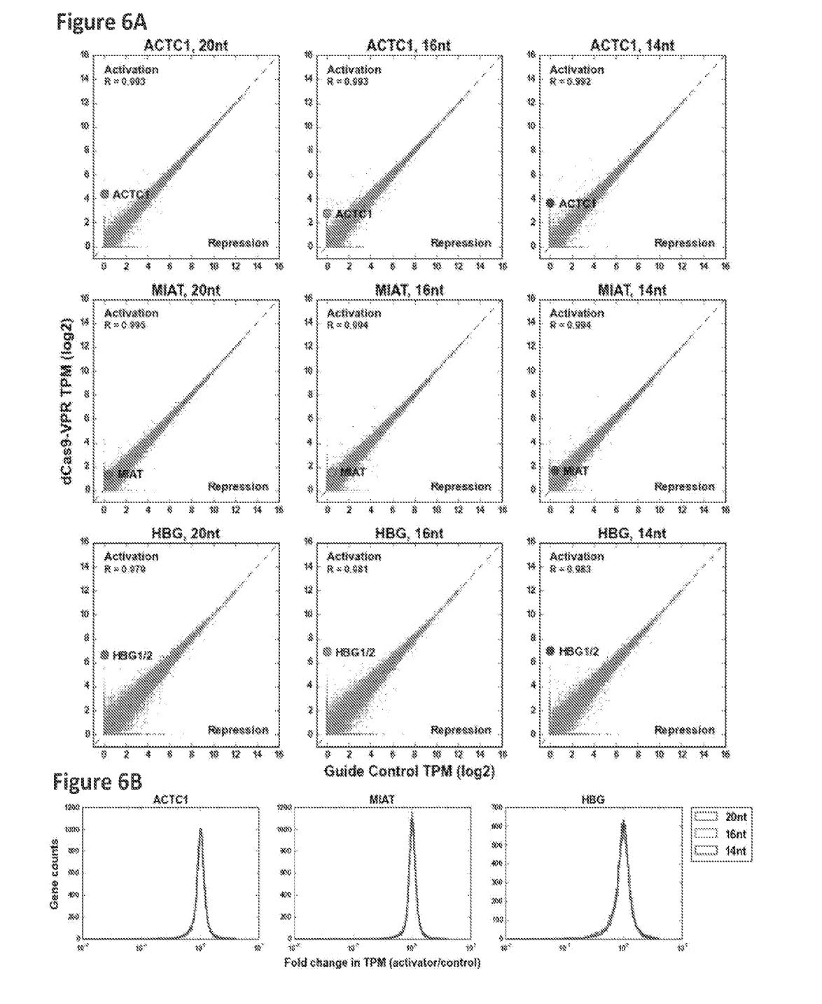

To characterize the difference in specificity between 14 and 20 nt gRNAs, three different reporter plasmids each with a unique gRNA binding site upstream of a fluorescent reporter gene were utilized. Overall 14 nt gRNAs showed a higher sensitivity (greater loss in reporter activation) when single mismatches were inserted into the gRNA, as compared to 20 nt gRNAs (see FIG. 5). To further probe the specificity of shortened gRNAs, 20, 16 and 14 nt gRNAs were expressed in combination with dCas9-VPR, and targeted either ACTC1, MIAT or HBG1. By high throughput RNA sequencing, a significant difference (p=0.316, one-way within-sample ANOVA) was not observed in the number of off-target changes in gene expression between 14, 16, and 20 nt guides, and overall correlation in gene expression between samples and controls remained high (R≥0.979) in all cases (see FIG. 6). These data as a whole suggest that utilizing 16 or 14 nt gRNAs does not lead to a significant increase in undesired off-target activity.

Accordingly, enzymatic activity and transcriptional activation of an enzymatically active Cas9-VPR fusion was modulated by altering the spacer sequence length of the guide RNA.

### EXAMPLE X

### Simultaneous Gene Editing and Transcriptional Activation

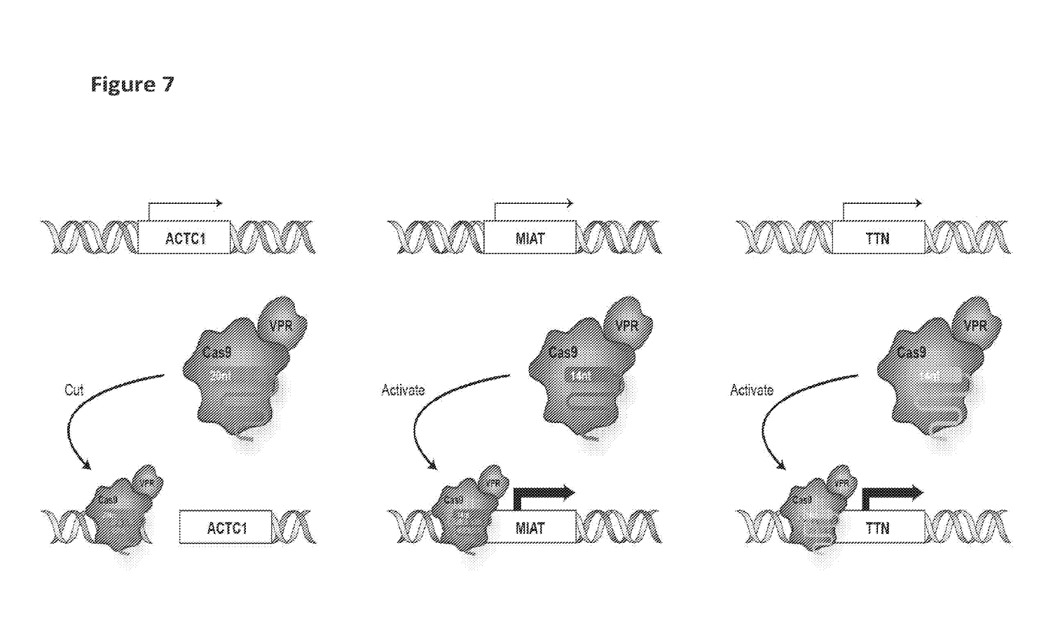

Cas9 nuclease activity was modulated by altering gRNA length. Nuclease-independent and nuclease-dependent functions were performed within a population of cells via introduction of a single Cas9 protein achieving multifunctional, multiplex genome engineering. Cas9 or Cas9-VPR was co-transfected along with a series of gRNAs targeting TTN and MIAT for activation using 14 nt gRNAs and ACTC1 for mutation by employing a 20 nt gRNA. Compared to wild-type Cas9, Cas9-VPR exhibited robust TTN and MIAT gene induction while also generating a comparable level of genomic mutation at the ACTC1 locus. See FIG. 1D and FIG. 7.

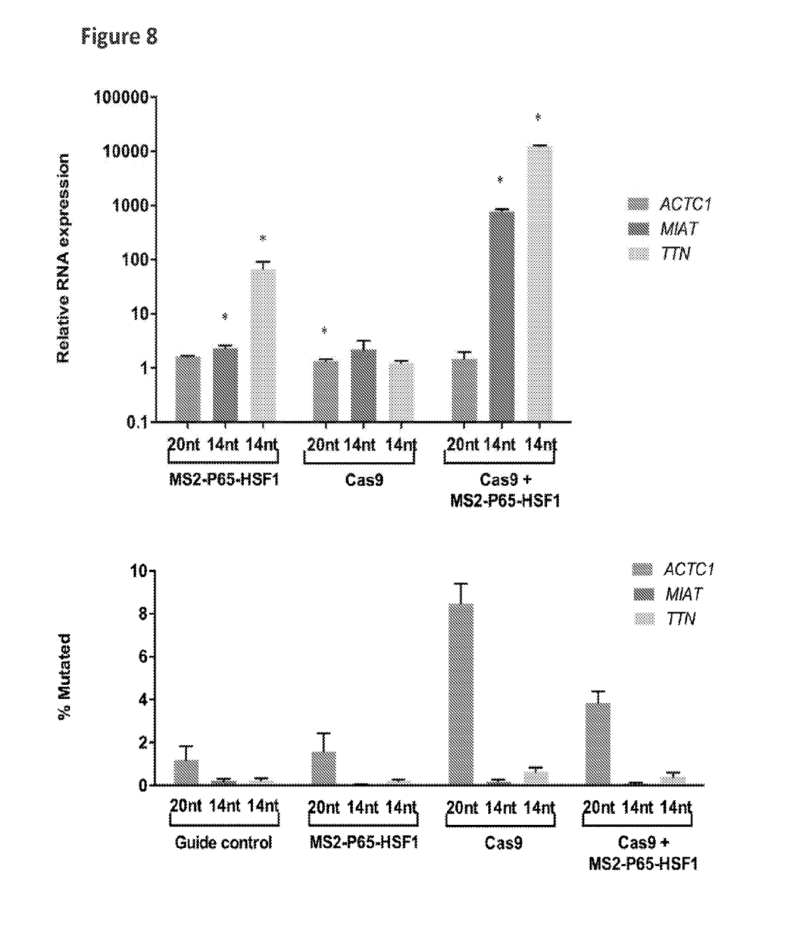

To enable multifunctional cellular engineering using Cas9-expressing cell lines and model organisms already developed by the scientific community, enzymatically active Cas9 attached to a transcriptional regulator was provided for concurrent cutting and activation using guide RNAs having different spacer sequence lengths. In one aspect, enzymatically active Cas9 was provided with transcriptional regulatory capacity by using a previously reported aptamer based activation system. See S. Konermann, M. D. Brigham, A. E. Trevino et al., Nature 517 (7536), 583 (2015). In this system, Cas9 mediated activation is not through direct fusion of an effector domain to Cas9, but rather via recruitment of the effector module through aptamers present in the gRNA. When tested, the aptamer-based activators enabled Cas9 to concurrently edit a genomic locus by using a standard 20 nt guide and induce the expression of a pair of target genes with 14 nt guides containing RNA aptamers. See FIG. 8.

### EXAMPLE XI

### Preventing Off-Target Mutations Using Truncated gRNAs

Aspects of the present disclosure are directed to methods of preventing or reducing the occurrence of off target mutations by using truncated guide RNAs as described herein. According to one aspect, given the ability of 16 nt or shorter gRNAs to enable Cas9 to bind to a target locus but not cut it, a system method or system is provided in which Cas9 is targeted to a desired locus via 25-17 nt gRNAs and at the same time a series of 16 to 8 nt gRNAs are simultaneously provided that are directed against each of the known Cas9 off-target sites. The 16 nt or shorter gRNAs would then bind the off-target regions and block them from being acted upon by the full length, for example, 20 nt gRNA, thus increasing on-target specificity. Accordingly, a method of reducing off site complex formation of an enzymatically active Cas9 to genomic DNA within a cell includes providing to the cell an enzymatically active Cas9, providing to the cell an enzymatic guide RNA complementary to a target nucleic acid sequence, providing to the cell a non-enzymatic guide RNA complementary to known off target binding sites for the enzymatically active Cas9, wherein the enzymatic guide RNA binds to the target nucleic acid and the Cas9 cleaves the target nucleic acid, wherein the non-enzymatic guide RNA bind to known off target binding sites for the enzymatically active Cas9 thereby inhibiting the enzymatic guide RNA from binding to the known off target binding sites.

### EXAMPLE XII

### Altering Strength of gRNA/Cas9/Target DNA Complex Formation

Aspects of the present disclosure are based on the discovery that different gRNA lengths have different binding strengths to a target nucleic acid. According to one aspect, the guide RNA length can be selected to provide a desired binding strength to a target nucleic acid. In this manner, by modulating the length of the gRNA, strength of Cas9-DNA interactions is controlled. Accordingly, a method is provided of forming two or more complexes of guide RNA and Cas9 at corresponding target nucleic acids where the two or more complexes of guide RNA and Cas9 have different binding strengths to their respective target nucleic acids based on differing guide RNA lengths.

Accordingly, a method of forming a plurality of guide RNA/Cas9 complexes at a plurality of target nucleic acid locations includes providing to the cell a Cas9, providing to the cell a plurality of guide RNA sequences of varying lengths complementary to corresponding target nucleic acid sequences wherein each guide RNA sequence has a different nucleotide length corresponding to a different binding strength, wherein the plurality of guide RNA sequences and the Cas9 co-localize at the corresponding target nucleic acids to form a complex, where each complex has a different binding strength to the target nucleic acid.

In accordance with an alternate aspect, selection of a guide RNA length to promote binding of a nuclease null Cas9 or an enzymatically active Cas9 but rendered nuclease null by the guide RNA length is provided to inhibit expression of one allele of a gene while not inhibiting expression of the other allele of the gene. According to this aspect, a first allele of a gene includes a greater number of repeat sequences compared to a second allele of a gene. By selecting a guide RNA of sufficient length to bind at a desired binding strength to the greater number of repeat sequences of the first allele, the combined binding at the selected binding strength is sufficient to inhibit expression of the first allele, while the binding of the guide RNA at the selected binding strength to the fewer repeat sequences of the second allele is insufficient to inhibit expression of the second allele. For example, such Cas9 tuning may be of particular interest in repeat expansion diseases (such as but not limited to Fragile X disease, Huntington's disease, Spinal Cerebellar Atrophy Type 1, 2, 3, 6, 7, 12 and 17) where patient's possess two copies of the same gene, with one allele containing a pathologically long stretch of the same sequence repeated. These repeat expansion containing alleles have been found to cause disease and if silenced would rescue the disease phenotype. A series of progressively shortened gRNAs are used to preferentially silence the expanded allele over the wild type allele, which possesses far fewer repeat expansions and so will complex fewer Cas9 molecules and experience less transcriptional inhibition due to Cas9 binding.

### EXAMPLE XIII

### Improving Specificity of Guide RNA/Cas9 Complex Formation

According to one aspect, the specificity of guide RNA/Cas9 complex formation can be increased by using shorter guide RNA sequences compared to longer guide RNA sequences. A shorter guide RNA sequence has a decreased tolerance for binding when a mismatch is present between the guide RNA and the target locus. Accordingly, shorter guide RNA sequences are more specific to where they will bind. Selecting a shorter guide RNA sequence will decrease off target binding of the guide RNA because specificity will be increased for the target nucleic acid sequence. This improved specificity can be exploited to further enhance the targeting of nuclease null variants of Cas9 to a given region. Examples where this may be of use is in targeting two dCas9-Fokl variants to a desired genomic locus while further inhibiting off-target binding, or in conjunction with dCas9-activator or repressor constructs to assure only the targeted genomic region is affected.

In addition, aspects of the present disclosure are directed to methods of enriching for a desired target nucleic acid locus from a complex mixture of DNA fragments (either genomic, metagenomics or synthetic) using shorter guide RNA sequences, such as a guide RNA sequence having 16 nucleotides or fewer, such as a guide RNA sequence having between 16 nucleotides and 8 nucleotides in length. Aspects for this method are directed to increased specificity seen with shorter guide RNA, such as a guide RNA having 14 nucleotides or fewer. According to this aspect, a method is provided using guide RNA having 16 or fewer nucleotides in length to enrich for a given target sequence of interest over other off-target regions. Aspects of such a method are directed to specific enrichment of a single locus, building large libraries of gRNAs to perform targeted enrichment of hundreds of sites within any complex mixture of DNA be it from a single genome or metagenome, targeted exome capture, selectively binding a particular gene orthologue over others from a complex metagenomics sample, use of a single Cas9 protein to simultaneously perform both sequence fragmentation using full length 20 nt-17 nt gRNAs along with sequence capture using ≤16 nt gRNAs, such as 16 nt to 8 nt.

1. A method of modulating expression of a target nucleic acid in a cell comprising
providing to the cell an enzymatically active Cas9 optionally having a transcriptional regulator attached thereto,
providing to the cell a guide RNA having a spacer sequence of 14 to 8 nucleotides wherein the guide RNA and the enzymatically active Cas9 optionally having a transcriptional regulator attached thereto form a co-localization complex with the target nucleic acid and wherein complex formation of the cas9 and the guide RNA at the target nucleic acid regulates expression of the target nucleic acid or the transcriptional regulator, if present, regulates expression of the target nucleic acid.

2. The method of claim 1 wherein the enzymatically active Cas9 is fused to the transcriptional regulator.

3. The method of claim 1 wherein the enzymatically active Cas9 having a transcriptional regulator attached thereto is provided to the cell by introducing into the cell a first foreign nucleic acid encoding the enzymatically active Cas9 having a transcriptional regulator attached thereto and wherein the guide RNA is provided to the cell by introducing into the cell a second foreign nucleic acid encoding the guide RNA, wherein the first foreign nucleic acid and the second foreign nucleic acid are provided to the cell on the same or different vectors,
wherein the guide RNA and the enzymatically active Cas9 having a transcriptional regulator attached thereto are expressed,
wherein the guide RNA and the enzymatically active Cas9 having a transcriptional regulator attached thereto co-localize to the target nucleic acid.

4. The method of claim 1 wherein the enzymatically active Cas9 is an enzymatically active Cas9 nickase.

5. The method of claim 1 wherein the cell is in vitro, in vivo or ex vivo.

6. The method of claim 1 wherein the cell is a eukaryotic cell or prokaryotic cell.

7. The method of claim 1 wherein the cell is a bacteria cell, a yeast cell, a fungal cell, a mammalian cell, a plant cell or an animal cell.

8. The method of claim 1 wherein the target nucleic acid is genomic DNA, mitochondrial DNA, plastid DNA, viral DNA, or exogenous DNA.

9. The method of claim 1 wherein enzymatically active Cas9 does not cut or nick the target nucleic acid.

10. A cell comprising
an enzymatically active Cas9 having a transcriptional regulator attached thereto and
a guide RNA having a spacer sequence of 14 to 8 nucleotides and wherein the guide RNA and the enzymatically active Cas9 are members of a co-localization complex for a target nucleic acid.

11. The cell of claim 10 wherein the cell is a eukaryotic cell or prokaryotic cell.

12. The cell of claim 10 wherein the cell is a bacteria cell, a yeast cell, a fungal cell, a mammalian cell, a plant cell or an animal cell.

13. A cell comprising
a first foreign nucleic acid encoding an enzymatically active Cas9 having a transcriptional regulator attached thereto and
a second foreign nucleic acid encoding a guide RNA having a spacer sequence of 14 to 8 nucleotides and wherein the guide RNA and the enzymatically active Cas9 having a transcriptional regulator attached thereto are members of a co-localization complex for the target nucleic acid.

14. The cell of claim 13 wherein the cell is a eukaryotic cell or prokaryotic cell.

15. The cell of claim 13 wherein the cell is a bacteria cell, a yeast cell, a fungal cell, a mammalian cell, a plant cell or an animal cell.

16. A method of modulating expression of a first target nucleic acid in a cell and altering expression of second target nucleic acid in the cell comprising
providing to the cell an enzymatically active Cas9 having a transcriptional regulator attached thereto,
providing to the cell a first guide RNA having a spacer sequence of 14 to 8 nucleotides,
providing to the cell a second guide RNA having a spacer sequence of 25 to 15 nucleotides,
wherein the first guide RNA and the enzymatically active Cas9 having a transcriptional regulator attached thereto form a co-localization complex with the first target nucleic acid and wherein the transcriptional regulator regulates expression of the target nucleic acid, and
wherein the second guide RNA and the enzymatically active Cas9 having a transcriptional regulator attached thereto form a co-localization complex with the second target nucleic acid and wherein the enzymatically active Cas9 cleaves the second target nucleic acid in a site specific manner.

17. The method of claim 16 wherein the first guide RNA is provided to the cell before the second guide RNA is provided to the cell.

18. The method of claim 16 wherein the second guide RNA is provided to the cell before the first guide RNA is provided to the cell.

19. The method of claim 16 wherein the first guide RNA and the second guide RNA are provided to the cell simultaneously.

20. The method of claim 16 wherein the first target nucleic acid and the second target nucleic acid are different.

21. The method of claim 16 wherein the first target nucleic acid and the second target nucleic acid are the same.

22. The method of claim 16 wherein the enzymatically active Cas9 is fused to the transcriptional regulator.

23. The method of claim 16 wherein the enzymatically active Cas9 having a transcriptional regulator attached thereto is provided to the cell by introducing into the cell a first foreign nucleic acid encoding the enzymatically active Cas9 having a transcriptional regulator attached thereto, wherein the first guide RNA is provided to the cell by introducing into the cell a second foreign nucleic acid encoding the first guide RNA, wherein the second guide RNA is provided to the cell by introducing into the cell a third foreign nucleic acid encoding the second guide RNA, wherein the first foreign nucleic acid, the second foreign nucleic acid, and the third foreign nucleic acid are provided to the cell on the same or different vectors,
wherein the first guide RNA, the second guide RNA and the enzymatically active Cas9 having a transcriptional regulator attached thereto are expressed,
wherein the first guide RNA and the enzymatically active Cas9 having a transcriptional regulator attached thereto co-localize to the first target nucleic acid, and
wherein the second guide RNA and the enzymatically active Cas9 having a transcriptional regulator attached thereto co-localize to the second target nucleic acid.

24. The method of claim 16 wherein the enzymatically active Cas9 is an enzymatically active Cas9 nickase.

25. The method of claim 16 wherein the cell is in vitro, in vivo or ex vivo.

26. The method of claim 16 wherein the cell is a eukaryotic cell or prokaryotic cell.

27. The method of claim 16 wherein the cell is a bacteria cell, a yeast cell, a fungal cell, a mammalian cell, a plant cell or an animal cell.

28. The method of claim 16 wherein the target nucleic acid is genomic DNA, mitochondrial DNA, plastid DNA, viral DNA, or exogenous DNA.

29. A cell comprising
an enzymatically active Cas9 having a transcriptional regulator attached thereto,
a first guide RNA having a spacer sequence of 14 to 8 nucleotides, and wherein the guide RNA and the enzymatically active Cas9 are members of a co-localization complex for a first target nucleic acid, and
a second guide RNA having a spacer sequence of 25 to 15 nucleotides, and wherein the second guide RNA and the enzymatically active Cas9 protein are members of a co-localization complex for a second target nucleic acid.

30. The cell of claim 29 wherein the cell is a eukaryotic cell or prokaryotic cell.

31. The cell of claim 29 wherein the cell is a bacteria cell, a yeast cell, a fungal cell, a mammalian cell, a plant cell or an animal cell.

32. A cell comprising
a first foreign nucleic acid encoding an enzymatically active Cas9 having a transcriptional regulator attached thereto,
a second foreign nucleic acid encoding a first guide RNA having a spacer sequence of 14 to 8 nucleotides and wherein the first guide RNA and the enzymatically active Cas9 having a transcriptional regulator attached thereto are members of a co-localization complex for a first target nucleic acid, and
a third foreign nucleic acid encoding a second guide RNA having a spacer sequence of 25 to 15 nucleotides and wherein the second guide RNA and the enzymatically active Cas9 having a transcriptional regulator attached thereto are members of a co-localization complex for a second target nucleic acid.

33. The cell of claim 32 wherein the cell is a eukaryotic cell or prokaryotic cell.

34. The cell of claim 32 wherein the cell is a bacteria cell, a yeast cell, a fungal cell, a mammalian cell, a plant cell or an animal cell.
